(19) United States
(12) Patent Application Publication
(10) Pub. No.: US 2012/0310111 A1
(43) Pub. Date: Dec. 6, 2012

(54) MAGNETIC LINEAR ACTUATOR FOR DEPLOYABLE CATHETER TOOLS

(75) Inventors: Yehoshua Shachar, Santa Monica, CA (US); Leslie Farkas, Ojai, CA (US)

(73) Assignee: Magnetecs, Inc., Inglewood, CA (US)

(21) Appl. No.: 13/464,091

(22) Filed: May 4, 2012

Related U.S. Application Data
(63) Continuation of application No. 12/103,518, filed on Apr. 15, 2008, now abandoned.
(60) Provisional application No. 60/940,941, filed on May 30, 2007.

(51) Int. Cl. A61M 25/092; A61B 10/02

(52) U.S. Cl. 600/567; 604/95.01

(57) ABSTRACT

Using the linear forces that are provided by an electromagnetic solenoid applied near the distal end of a medical catheter, various surgical instruments can be actuated or deployed for use in interventional medicine. The linear actuator uses the principles of magnetic repulsion and attraction as a means for moving a bobbin that can be attached to various types of moving components that translate linear movements into the actuation of a tool that is attached to the linear actuator. Using independent solenoid coils, movement modality is increased from two possible positions to three.

### REFERENCE TO RELATED APPLICATIONS

This application is a continuation of U.S. patent application Ser. No. 12/103,518, filed Apr. 15, 2008, which claims priority to U.S. Provisional Application No. 60/690,941, filed on May 30, 2007, titled “LINEAR ACTUATED CATHETER TOOLS,” the entire contents of both of which are hereby incorporated by reference.

### BACKGROUND

1. Field of the Invention

The invention relates to the field of mechanical deployment and actuation of minimally invasive medical catheter tools by the transfer of electromagnetic forces into linear mechanical motion.

2. Description of the Related Art

Interventional medicine is the collection of medical procedures in which access to the treatment area is made by navigating through a patient's blood vessels, body cavities, or lumens.

Minimally invasive technologies have long been applied to surgical instruments such as pliers, forceps, and shears, and are applied to a variety of medical procedures.

Prior art actuators have traditionally used the transfer of mechanical forces applied to the proximal end of the tool in order to actuate or engage the working end, or distal end of the tool. Prime examples of this can be found in U.S. Pat. Nos. 6,551,302 (“Rosinko”) and 7,229,421 (“Jen”) where energy used in the mechanical rotation of an inner deflection knob or inner key becomes translated into linear motion by the actuator. The linear motion produced by the actuator is then used to operate or activate the medical tool located on the distal end of the catheter.

Other presently available interventional devices include robotically controlled actuators which provide the physician with greater precision and control of the applied forces that are used while performing a desired action.

While the catheter and magnetic actuators presented above have had successes in their respective fields, they are not without their drawbacks and limitations, particularly when it comes to the field of medicine.

In actuators that are used on medical catheters by providing power to an actuator by manually rotating a handle, the actuator procedure is open to human error and can lead to imprecise tool activation or other errors. Additionally, in a situation where a magnetic invasive surgery takes place, it can be cumbersome and inefficient for an operating physician to manually active an actuator while also trying to avoid bumping into or hitting other equipment such as electromagnets, and any other medical apparatuses at the same time.

Magnetic actuators for use in liquid or gas pipelines or in construction work have not been envisioned to work within the limited space that is available on a medical catheter. Nothing in the prior art suggests that a magnetic actuator may be reduced in size and specifically adapted for operating a medical tool located on the distal tip of a catheter for use in a invasive surgery.

### SUMMARY

These and other problems are solved by a linear actuator that is magnetically controlled and specifically designed to be placed on a medical catheter and work with an entire multitude of medical tools, thus giving the operating physician greater control and precision of his medical instruments with less possibility for error or mistake.

Using the linear forces that are provided by an electromagnetic solenoid applied near the distal end of a medical catheter, various surgical instruments can be actuated or deployed for use in interventional medicine. The linear actuator uses the principles of magnetic repulsion and attraction to produce forces for moving a bobbin that can be attached to various types of moving components that translate linear movements into the actuation of a tool that is attached to the linear actuator. Using independent solenoid coils, movement modality is increased from two possible positions to three or more.

A solenoid that contains several windings or turns of wire wrapped around it. The solenoid is a coil of wire designed to create a sufficiently strong magnetic field inside of the coil. By wrapping the same wire many times around cylinder, the magnetic field produced by the wires can become quite strong. The number of turns N refers to the number of loops the solenoid has. More loops will bring about a stronger magnetic field. Ampere's law can be applied to find the magnetic field inside of a long solenoid as a function of the number of turns per unit length, N/L, and the current I as shown in equation (1):

$${\sum }_{i} {B}_{i} Δ {L}_{i} cos {θ}_{i} = {μ}_{0} I ⇒ B \cdot x = {μ}_{0} ( \frac{N}{L} x ) I \qquad ( 1 )$$

The term (N/L)x represents the number of loops enclosed by the path. Only the upper portion of the path contributes to the sum because the magnetic field is zero outside the solenoid and because the vertical paths are perpendicular to the magnetic field and thus do not contribute. By dividing x out of both sides of equation (1), one finds:

$$B = {μ}_{0} ( \frac{N}{L} ) I \qquad ( 2 )$$

The magnetic field inside a solenoid is proportional to both the applied current and the number of turns per unit length. There is no dependence on the diameter of the solenoid or even on the shape of the solenoid. More importantly, the magnetic field is relatively constant inside the solenoid which means that any path placed within the solenoid will receive substantially the same amount of magnetic flux.

In one embodiment, the described solenoid winding is also wrapped around a bobbin which in turn is placed around a cylindrical rare earth permanent magnet with a predetermined size and length. The magnet has a hollow core so as to facilitate the passage of liquids to and from the catheter. The bobbin used is shorter than the permanent magnet and is free to slide along the magnet surface.

The coil creates a magnetic field which drives flux through the magnet, around the bobbin of the solenoid, through an air gap, and then back into the magnet. The reluctance of this path is mostly made up by the air gap. When the bobbin is off center to the magnet, the air gap is wide so the reluctance is quite high and the inductance is low. However, when a current is applied to the coil, the bobbin moves in the direction where reluctance of the circuit is reduced. The formulas for coil inductance and coil impedance are given in equations (3) and (4) respectively below:

$$\begin{matrix}L = \frac{{N}^{2}}{R} & ( 3 ) \\ X = \sqrt{{R}^{2} + {( 2 π fL )}^{2}} & ( 4 )\end{matrix}$$

The current that is driven through the coil is the voltage divided by the impedance given in equation (5) below:

$$I = \frac{V}{X} \qquad ( 5 )$$

In one embodiment, each solenoid has its own independent interconnecting wires which are connected to an outside power source. In one embodiment, one or more common wires are shared by one or more coils. This configuration allows electric currents to be driven in opposite directions within each solenoid and provides the necessary opposing magnetic flux for bringing the bobbin back to its original position and completes the movement of the medical tool.

When an electric current is applied from the outside source through each solenoid, a uniform magnetic field is produced which pushes or pulls the magnet in a predetermined linear direction. Coupled to the magnet is a small actuator arm which in turn is coupled by way of a series of hinges and pins to any variety of working tools such as jaws or clamps, needles, blades, or mapping and ablation probes.

In one embodiment with an actuated set of jaws or forceps is summarized further. For example, when an electric current is sent through the solenoid, a magnetic flux is created which pushes the magnet back towards the proximal end of the catheter. The actuator arm that is coupled to the magnet which had been set at an angle within the device is then straightened out until it is nearly parallel to the longitudinal axis of the catheter. The straightening of the actuator arm pulls on the upper jaw proximally, rotating the upper jaw about a central hinge in a clockwise direction and effectively opening the jaws. When the jaws are closed, the electric current in the solenoid is reversed in direction thus changing the direction of the magnetic flux and pushing the magnet back towards the distal end of the catheter. The actuator arm is then placed back into its original position and the upper jaw rotates counterclockwise around on the central hinge until it comes into contact with the sample tissue or the lower jaw portion of the device.

Using Maxwell's equations, the electromechanical force can be calculated using equation (6):

F=(Fₘ)²μ₀A/(2 g²) (6)

Equation (6) is used to calculate that for 7 to 12 French size catheters, 35 grams (or more) of constant force with a peak of 55 grams of force (or more) can be produced. Additional force can be produced by increasing the number of turns in the coil, by increasing the current, and/or increasing the strength of the permanent magnet.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

### BRIEF DESCRIPTION OF THE DRAWINGS

FIG. 1 is a longitudinal cross-section of the solenoid actuator.

FIG. 2 is a horizontal cross-section of the solenoid actuator.

FIG. 3 is a schematic representation of the solenoid actuator.

FIG. 4A is plan view of the magnetic coils when the actuator tool is in the full forward position.

FIG. 4B is a plan view of the magnetic coils when the actuator tool is in the mid position.

FIG. 4C is a plan view of the magnetic coils when the actuator tool is in the full back position.

FIG. 5A is a side and ¾ angle plan view of the device with the actuator tool in the full forward position.

FIG. 5B is a side and ¾ angle plan view of the device with the actuator tool in the full back position.

FIG. 6A is a plan view of an embodiment of the device comprising a blade or cutting tool at the distal tip of the catheter.

FIG. 6B is side plan view depicting the cutting tool embodiment of the device in the open and closed positions.

FIG. 6C is a plan view of the cutting tool embodiment of the device with the actuator sheathing pulled back.

FIG. 6D is a semi-exploded view of the cutting tool embodiment of the device coupled to the distal tip of the catheter.

FIG. 6E is a longitudinal cross-section of the cutting tool embodiment of the device in the closed position.

FIG. 6F is a longitudinal cross-section of the cutting tool embodiment of the device in the open position.

FIG. 7A is a plan view of an embodiment of the device comprising a set of jaws or clamps at the distal tip of the catheter.

FIG. 7B is a side plan view depicting the jaws or clamps embodiment of the device in the open and closed positions.

FIG. 7C is a plan view of the jaws or clamps embodiment of the device with the actuator sheathing pulled back.

FIG. 7D is a semi-exploded view of the jaws or clamps embodiment of the device coupled to the distal tip of the catheter.

FIG. 7E is a longitudinal cross-section of the jaws or clamps embodiment of the device in the closed position.

FIG. 7F is a longitudinal cross-section of the jaws or clamps embodiment of the device in the open position.

FIG. 8A is an orthographic view of the magnetically-deployable biopsy tool.

FIG. 8B is an orthographic representation of the biopsy tool is in its deployed state.

FIG. 8C is an isometric view of the main components of the biopsy tool when the biopsy tool is in its nested state.

FIG. 8D is an enlarged isometric view of the main components of the biopsy tool when the biopsy tool is in its deployed state.

FIG. 8E is a longitudinal cross-section of the biopsy tool in its deployed state.

FIG. 8F is a longitudinal cross-section of the biopsy tool in its nested state.

FIG. 9 is a block diagram of one embodiment which incorporates the magnetically-controlled linear actuator tool into a magnetically-guided Catheter Guidance Control and Imaging (CGCI) system.

### DETAILED DESCRIPTION

In general, the linear actuator for the deployment of catheter tools uses the principles of magnetic repulsion and attraction to produce forces for moving a bobbin that is attached to various types of moving components that translate the linear movements of the bobbin into the actuation of a tool that is coupled to the linear actuator on the distal tip of the catheter. Using independent coils that are coupled around the solenoid at different points allows the movement modality to be increased from two possible positions to three or more.

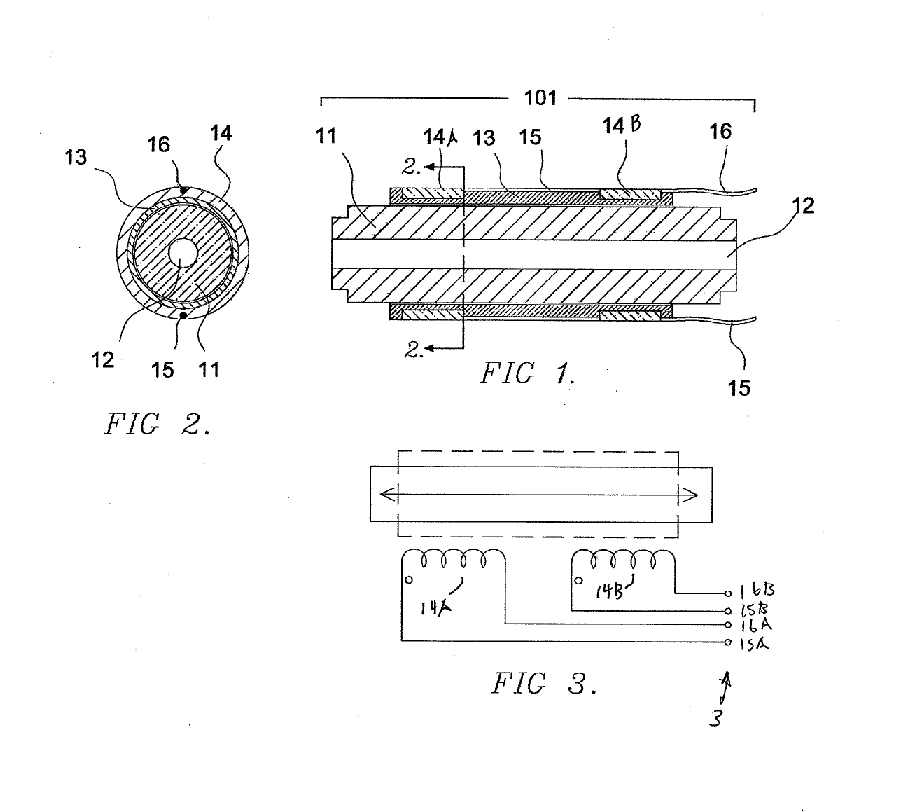

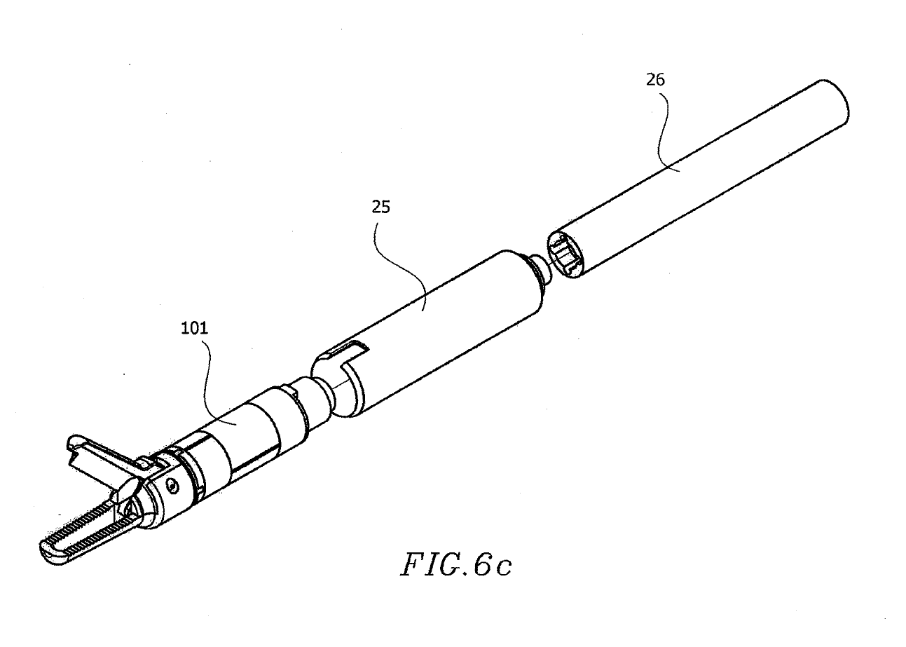

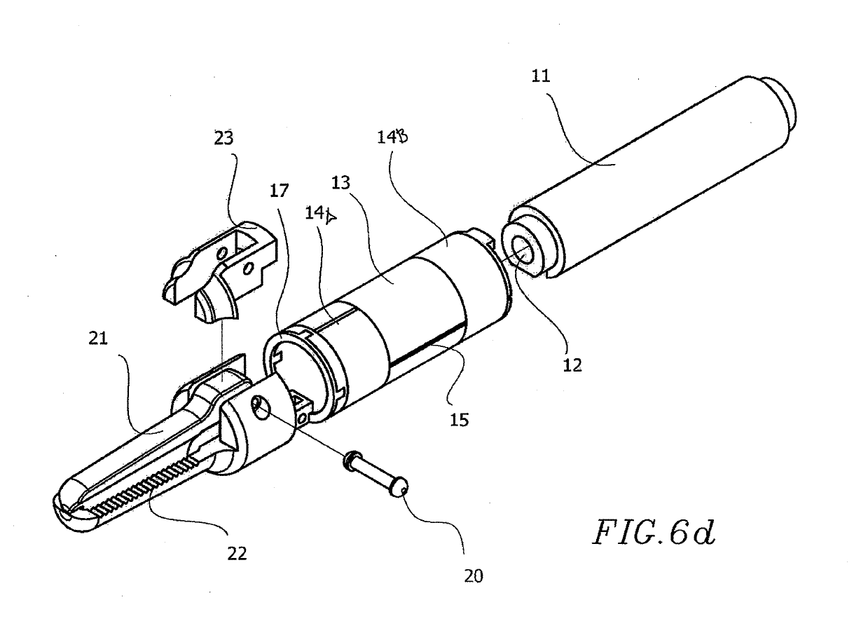

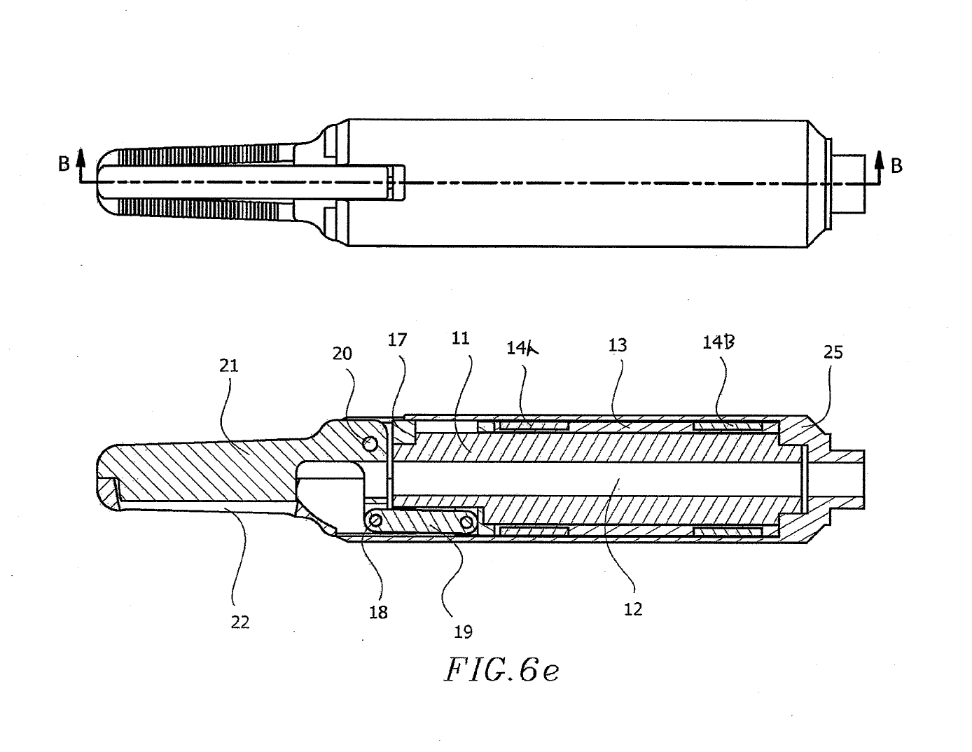

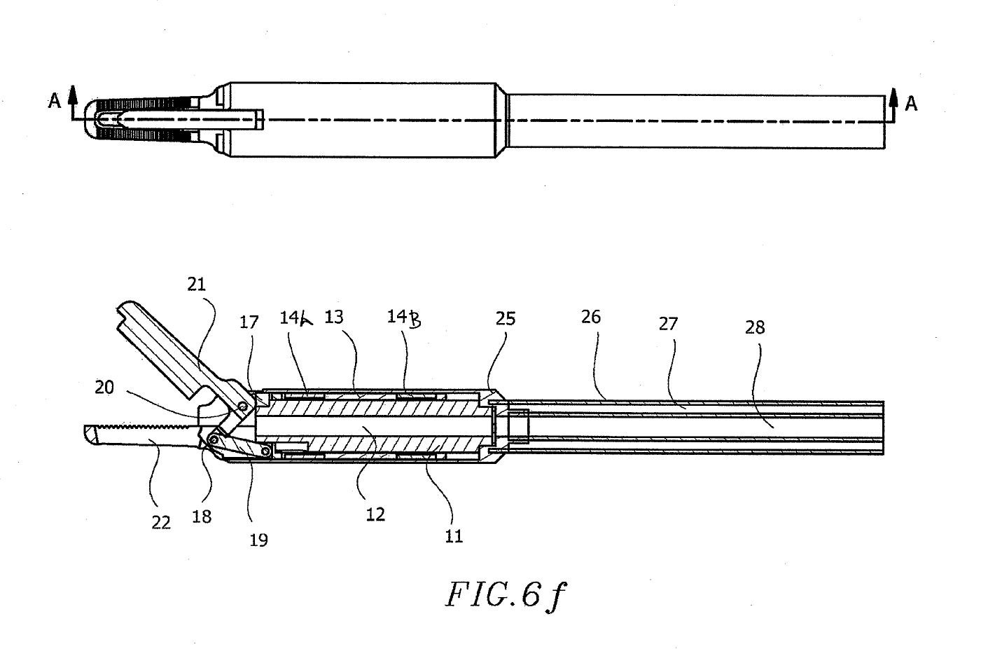

The magnetic linear actuator 101 as shown in FIG. 1 includes a first is a high coercive force permanent magnet 11 (e.g., made from Neodymium Iron Boron 48 MGOe or other magnet material) that is machined into a cylinder shape with a hollow core 12. The hollow core (also shown in FIG. 2) allows the passage of fluids to and from the catheter 26 (shown in FIG. 6A) when such a procedure is necessary. It is also expressly understood that the permanent magnet 11 may be made from any other suitable magnetic material or combination of magnetic materials.

FIG. 1 also shows a bobbin 13 and coil windings 14A and 14B (collectively coil windings 14). The bobbin 13 has two coil windings 14 placed close to its outer edge and each coil 14 is wound using 125 turns of 40 awg magnet wire, however, coils employing more turns or a different type of magnet wire may also be used. Each coil 14A,B has respective independent wires 15A,B and 16A,B coupled to them which allows for controlling the currents and their direction independently from each other. FIG. 3 shows each coil 14A,B independently connected to an outside power source via terminals 3. The coils 14A,B can also be connected to an outside power source by combining one or more of the wires 15A,B and 16A,B (for example, the wires 16A and 15B can be combined). Moreover, although two coils 14A and 14B are shown, three or more coils 14 can also be provided to further control the motion and/or position of the permanent magnet.

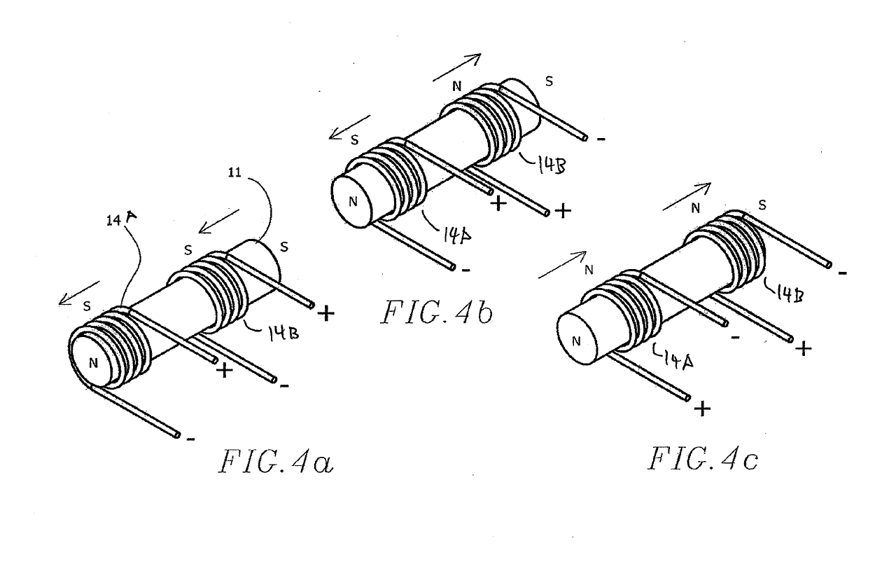

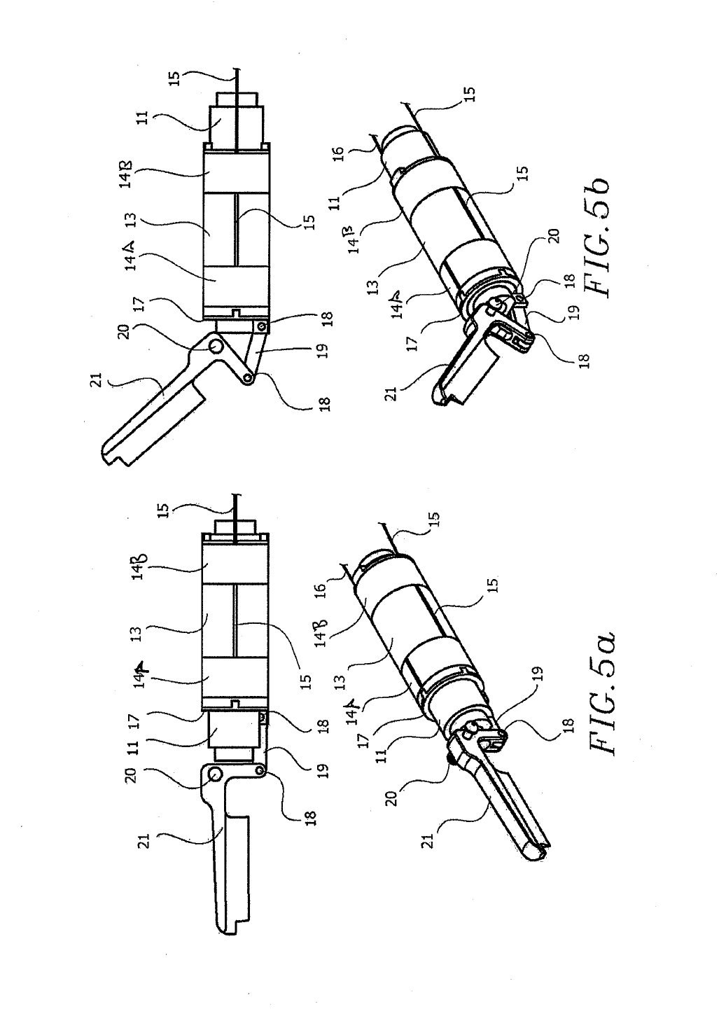

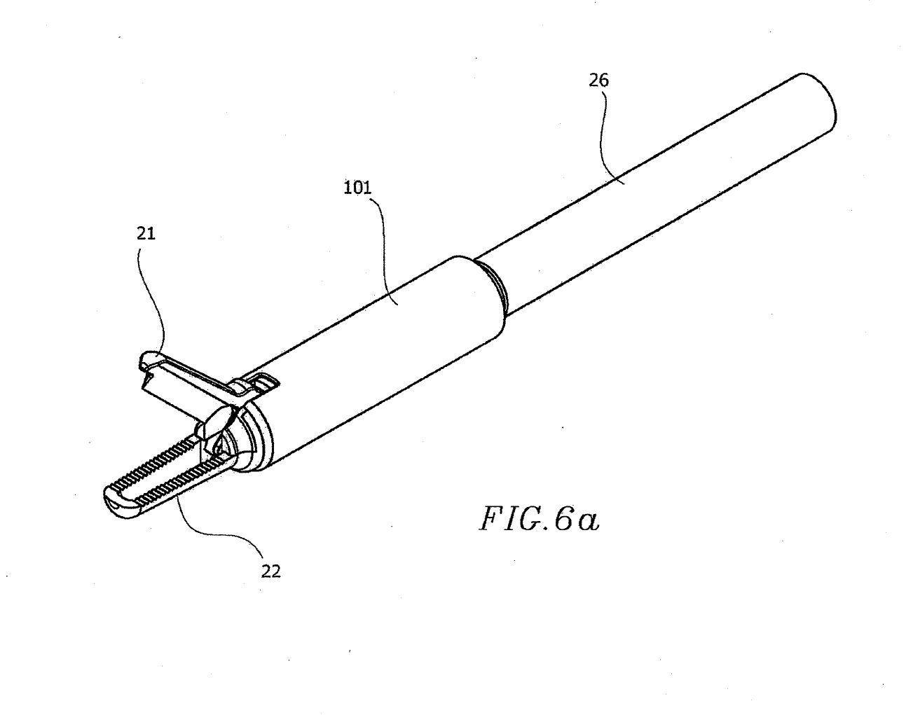

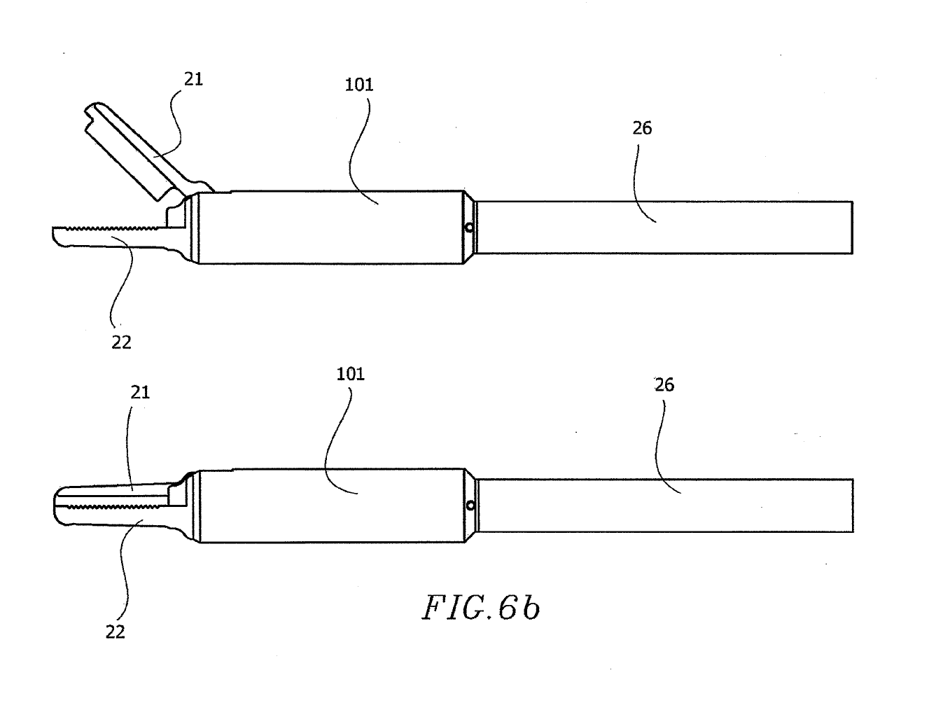

FIGS. 4A-4C shows the orientation and configuration of the coils 14 in each active position that the actuator tool passes through when the device is in use. For example, when the operating physician wishes to open or engage the medical tool located on the distal tip of the catheter, an electric current is sent in the same direction through both coils 14 as depicted in FIG. 4A. Having the current travel in the same direction in both coils 14 produces a strong magnetic flux in the same direction through the permanent magnet 11 which then by the law of superposition, pushes the magnet 11 towards the distal end of the catheter. A medical tool 21 is coupled to the actuator via a main fixed hinge pin 20 and a series of smaller hinge pins 18 coupled to an actuator arm 19. The actuator arm 19 is in turn coupled to a mechanical force transfer ring 17 which is coupled to the bobbin 13 and is free to slide along the surface of magnet 11. When the tool 21 is in the actuated position as depicted in FIG. 5B, the bobbin 13 and the mechanical force transfer ring 17 slide towards the distal end of the catheter and pushes the actuator arm 19 up against the medical tool 21. Because the medical tool 21 is held in place by the main fixed hinge pin 20, the actuator arm 19 can rotate about the smaller hinge pins 18 in order to compensate for the linear movement of the mechanical force transfer ring 17 and bobbin 13. As the actuator arm 19 continues to be pushed distally, the incoming rotational torque coming from the arm 19 is transferred to the medical tool 21 and causes it to begin rotating about the main fixed hinge pin 20 which effectively “opens” the medical tool 21. The medical tool 21 continues to open as long as the actuator arm 19 applies a rotational torque or until the main fixed hinge pin 20 has rotated to its maximum. The medical tool 21, the main fixed hinge pin 20, the actuator arm 19, the smaller hinge pins 18, and the mechanical force transfer ring 17 are, in one embodiment, made out of titanium for its strength, medical durability, and magnetic inertness; however a similar material can be used.

The operating physician can manipulate the amount the tool is actuated by adjusting the amount of current that is sent through the wires or altering the direction in which the current travels. FIG. 4B shows an example in which each coil 14A,B has an opposite orientated yet equal amount of current traveling through it. This configuration thus produces two equal and opposite magnetic fluxes which push and pull on the magnet 11 respectively in equal amounts and causes the actuator 101 and the tool 21 (both shown in FIG. 6A) to stop and maintain its current position.

When the operating physician wishes to close or disengage the medical tool and return it to its original position as depicted in FIG. 5A, the current in each coil 14 is once again applied in equal magnitude in the same orientation, however this time in the opposite direction from when the tool was opened. This configuration as depicted in FIG. 4C, produces a strong superimposed magnetic flux in the opposite direction from the flux created by the configuration in FIG. 4A used to open the tool, and pulls the magnet 11 in the proximal direction which thus pulls the actuator arm 19 down and causes the medical tool 21 to rotate back around the main fixed hinge pin 20. This procedure effectively “closes” the tool 21 and returns it to its original starting position. This process can then be repeated by continuously adjusting the coil 14 currents as many times as is required by the operating physician or as the situation dictates.

FIG. 6A shows another embodiment where the tool deployed on the distal tip of the catheter 26 is a cutting tool. The cutting tool comprises both a cutting blade 21 and a gripping element 22 for holding on to the tissue to be cut. The gripping element 22 also provides a durable surface for the cutting blade 21 to work against which aides in the ease of cutting the tissue or other biological material to be operated on.

FIG. 6B shows the cutting tool when not in use and when it is being activated by the linear actuator 101. When there is no current running through the linear actuator 101, the cutting blade 21 remains closed and rests against the gripping element 22. However when an electric current is applied, the linear actuator 101 lifts the cutting blade 21 into an “open” position as depicted in the upper diagram. The cutting blade 21 may be opened as far as 45 degrees (or more) from the longitudinal axis which places the most distal tip of the blade 7.4 mm above the gripping element 22.

FIGS. 6C and 6D show the device in various stages of deconstruction. FIG. 6C depicts the device with the actuator assembly sheath 25 pulled back from the linear actuator 101. The actuator assembly sheath 25 is, in one embodiment, made of medical grade PVC. FIG. 6D further shows each component of the cutting tool 21 and its positional relationship to the various parts of the linear actuator 101 including the medical tool housing 23 which fully encloses the cutting tool 21 into the device. The medical tool housing 23 is, in one embodiment, made out of Teflon, but other materials (e.g., plastics, metals, etc.) can be used as well.

FIGS. 6E and 6F are longitudinal cross sections of the cutting tool 21 and linear actuator 101 in the “closed” or un-actuated position, and in the “open” or actuated position respectively. FIG. 6F additionally depicts that the catheter 26 has multiple lumens, namely wire tunnels 27 for housing the wires that apply electric current to the coils 14, and a fluid and vacuum tunnel 28 for transferring fluid to and from the device.

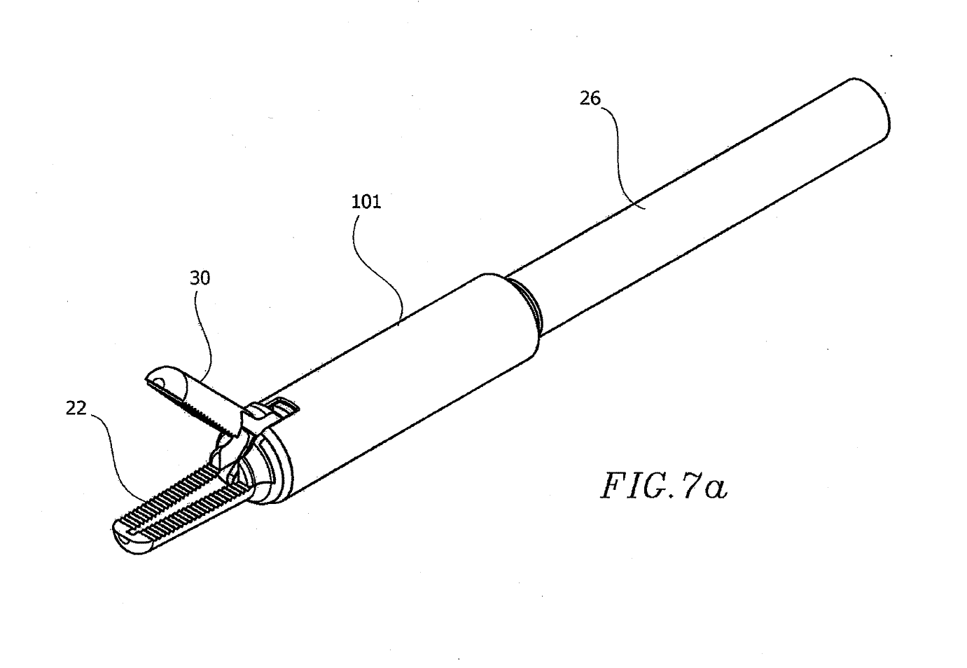

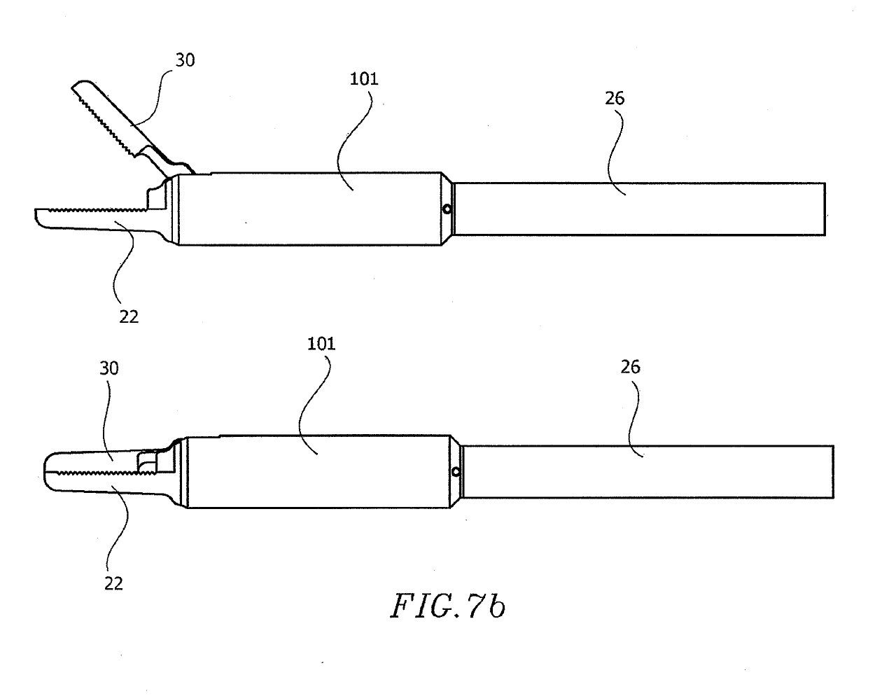

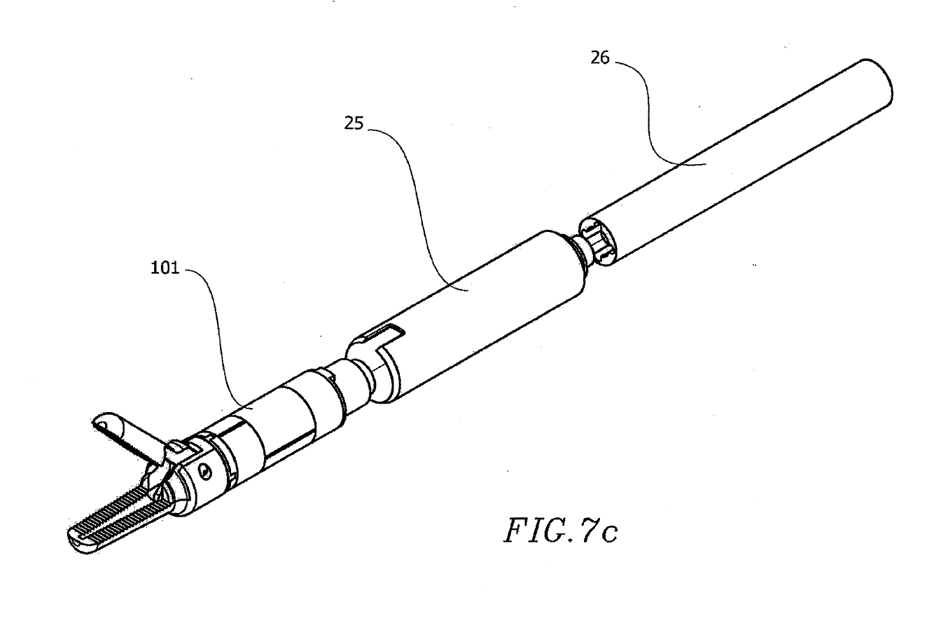

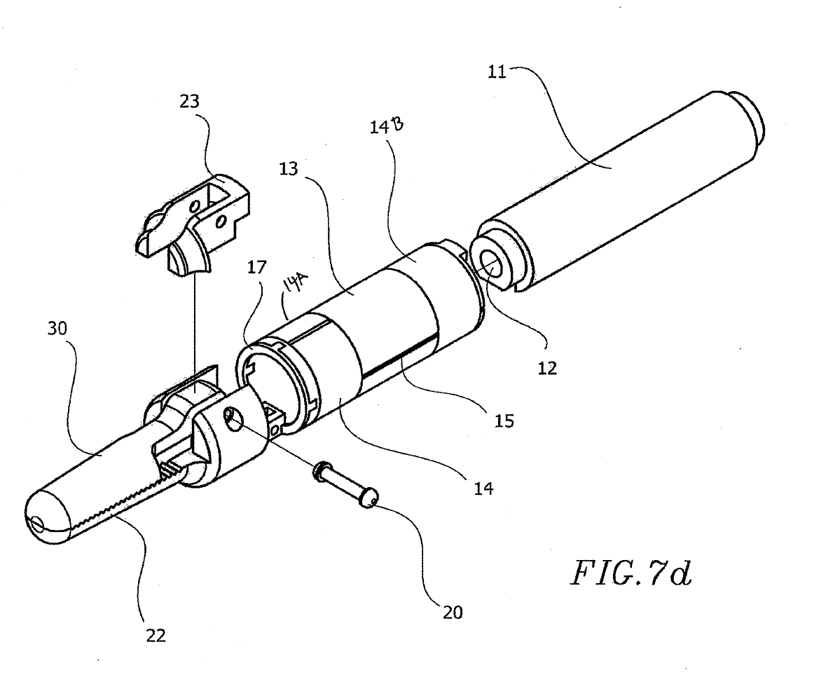

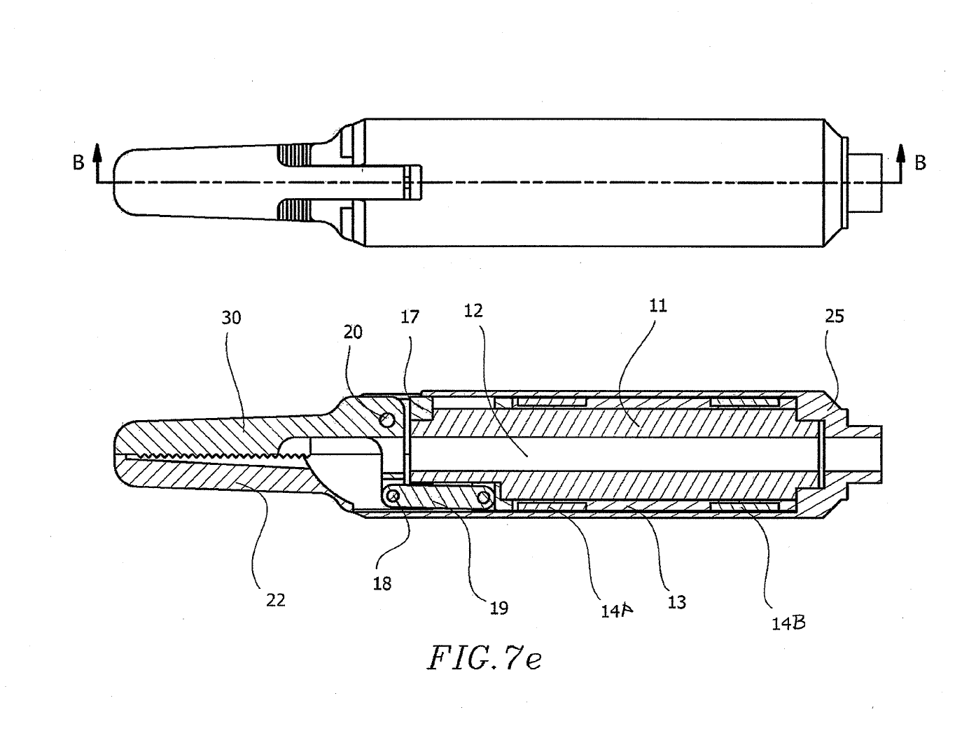

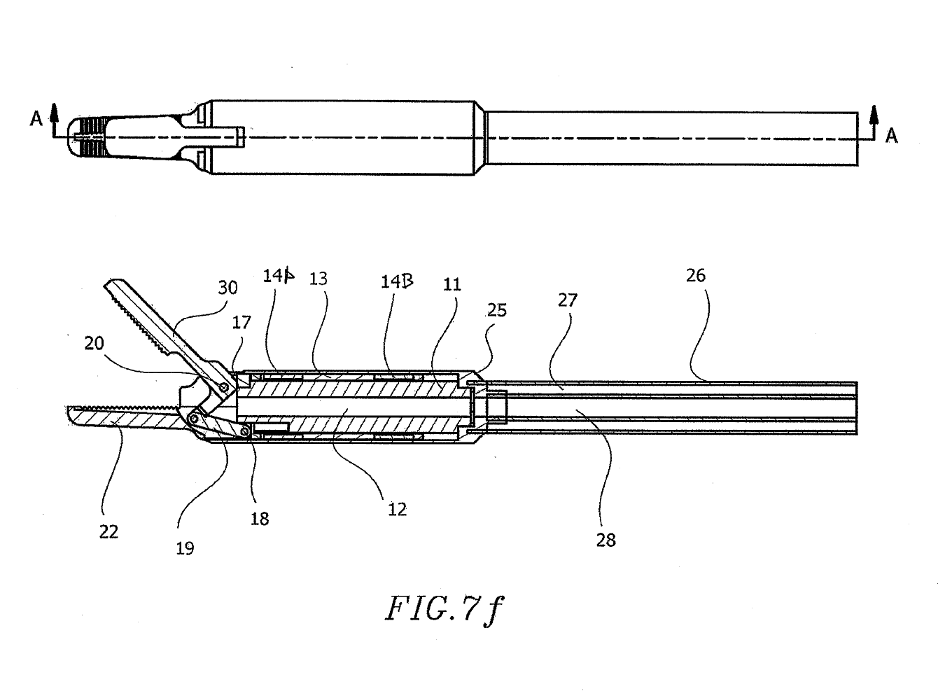

FIG. 7A shows another embodiment where the tool deployed on the distal tip of the catheter 26 is a forceps tool. The forceps tool comprises both an upper gripping element 30 and a lower gripping element 22 for holding on to the tissue or other biological material. The lower gripping element 22 also provides a durable surface for the upper gripping element 30 to work against which aides in the ease of gripping or holding the tissue or other biological material to be operated on.

FIG. 7B shows the forceps tool when not in use and when it is being activated by the linear actuator 101. When there is no current running through the linear actuator 101, the upper gripping element 30 remains closed and rest against the lower gripping element 22. However, when an electric current is applied, the linear actuator 101 lifts the upper gripping element 30 into an “open” position as depicted in the upper diagram. The upper gripping element 30 may be opened as far as 48 degrees from the longitudinal axis which places the most distal tip of the upper gripping element 30 a desired distance (9.34 mm in one embodiment) above the lower gripping element 22.

FIGS. 7C and 7D show the device in various stages of deconstruction. FIG. 7C depicts the device with the actuator assembly sheath 25 pulled back from the linear actuator 101. The actuator assembly sheath 25 is, in one embodiment, made of medical grade PVC. FIG. 7D further shows each component of the clamp tool 30 and its positional relationship to the various parts of the linear actuator 101 including the medical tool housing 23 which fully encloses the upper gripping element 30 into the device. The medical tool housing 23 is, in one embodiment, made out of Teflon.

FIGS. 7E and 7F are longitudinal cross sections of the upper gripping element 30 and linear actuator 101 in the “closed” or un-actuated position, and in the “open” or actuated position respectively. FIG. 7F additionally depicts that the catheter 26 has multiple lumens, namely wire tunnels 27 for housing the wires that apply electric current to the coils 14, and a fluid and vacuum tunnel 28 for transferring fluid to and from the device.

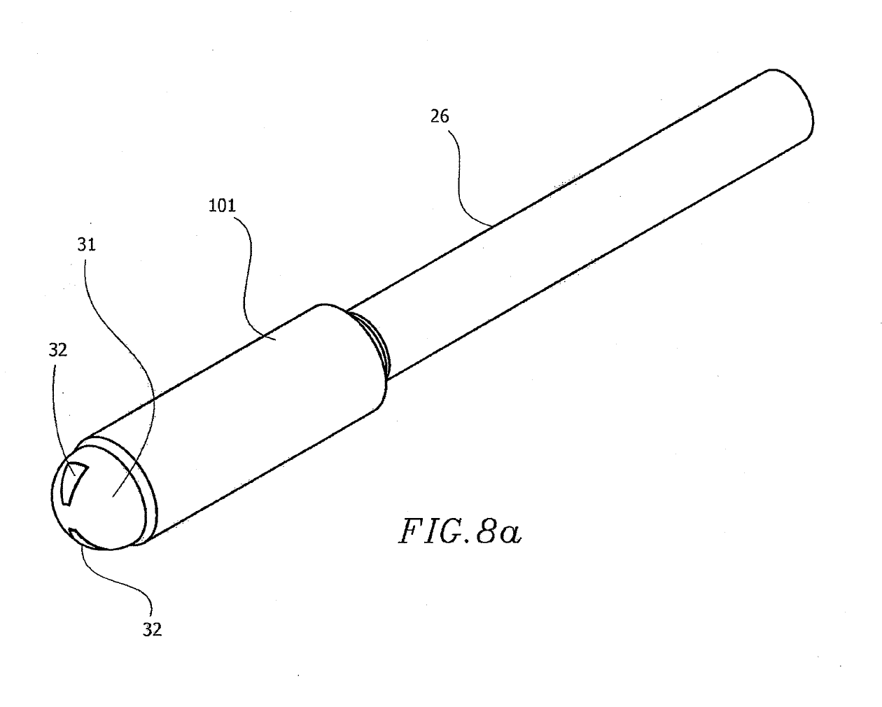

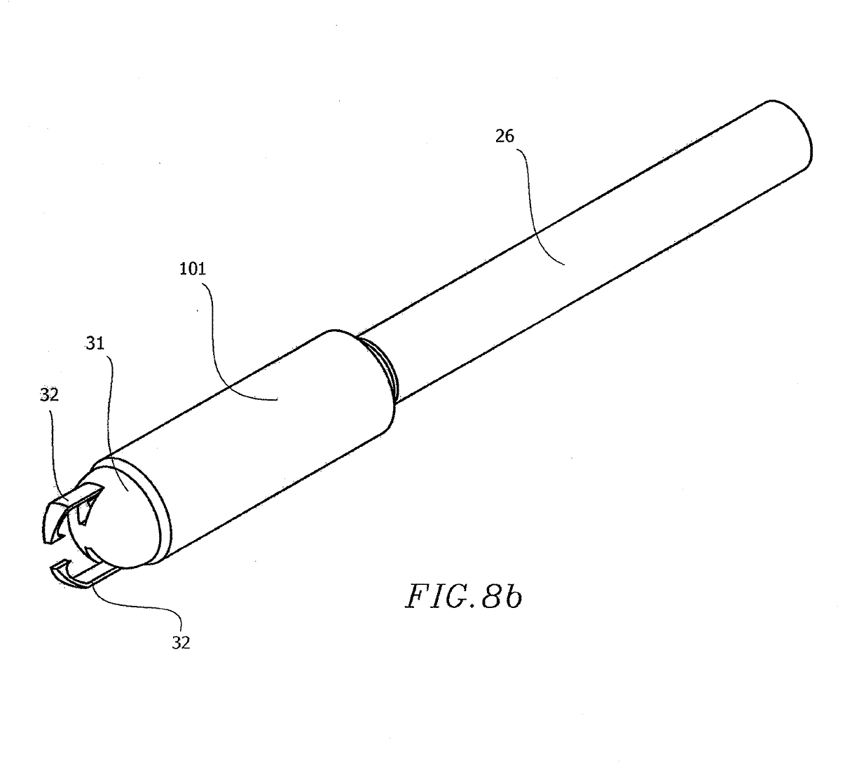

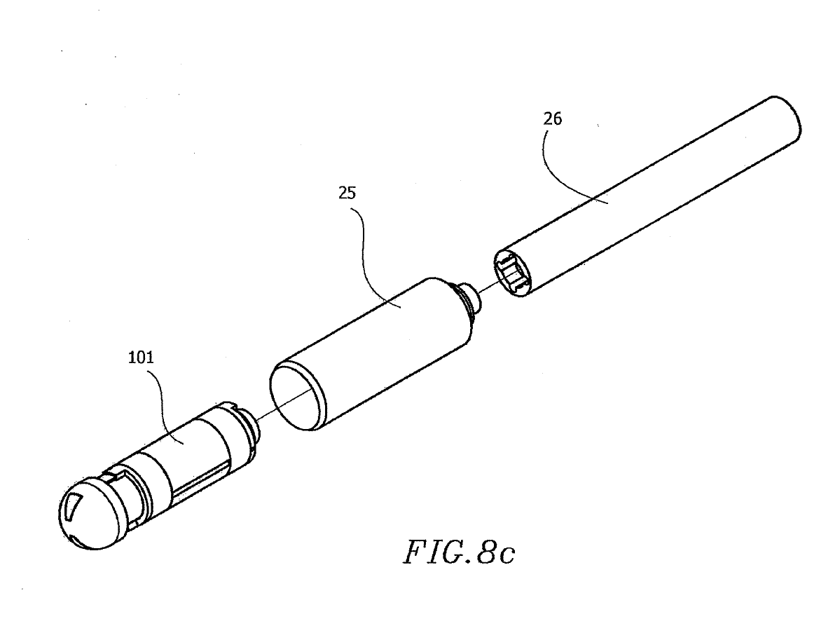

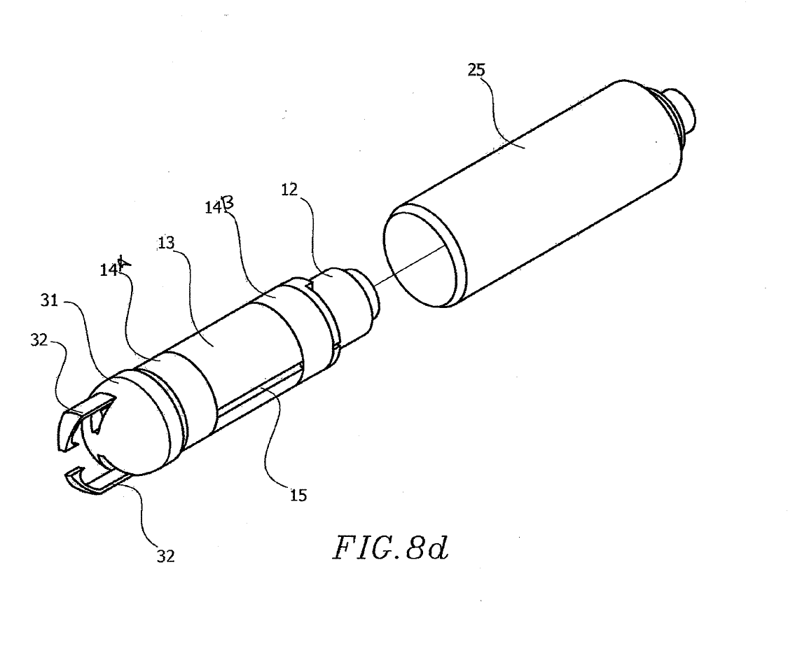

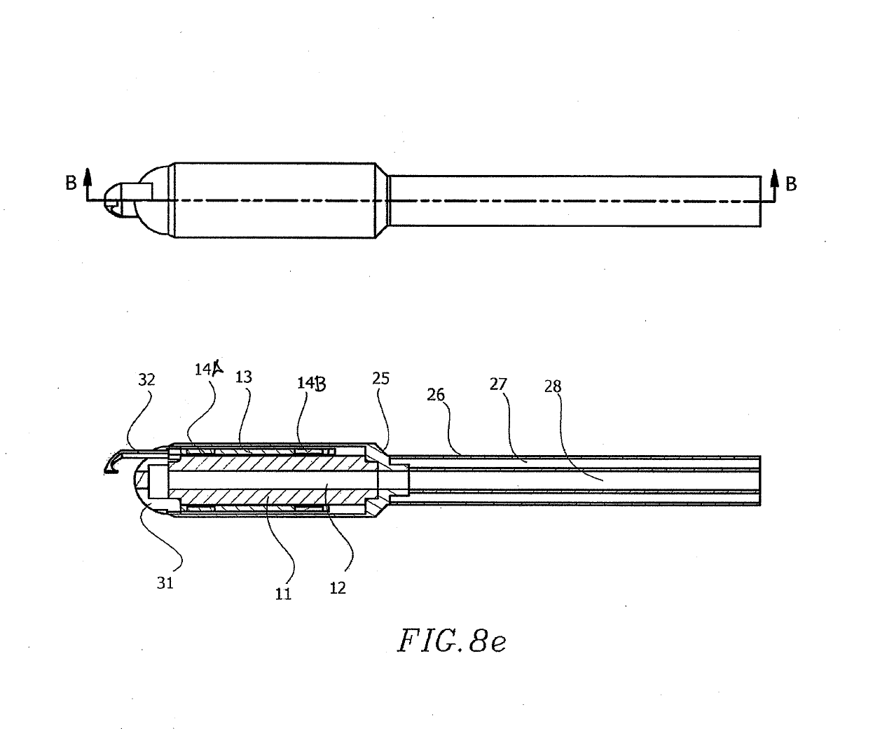

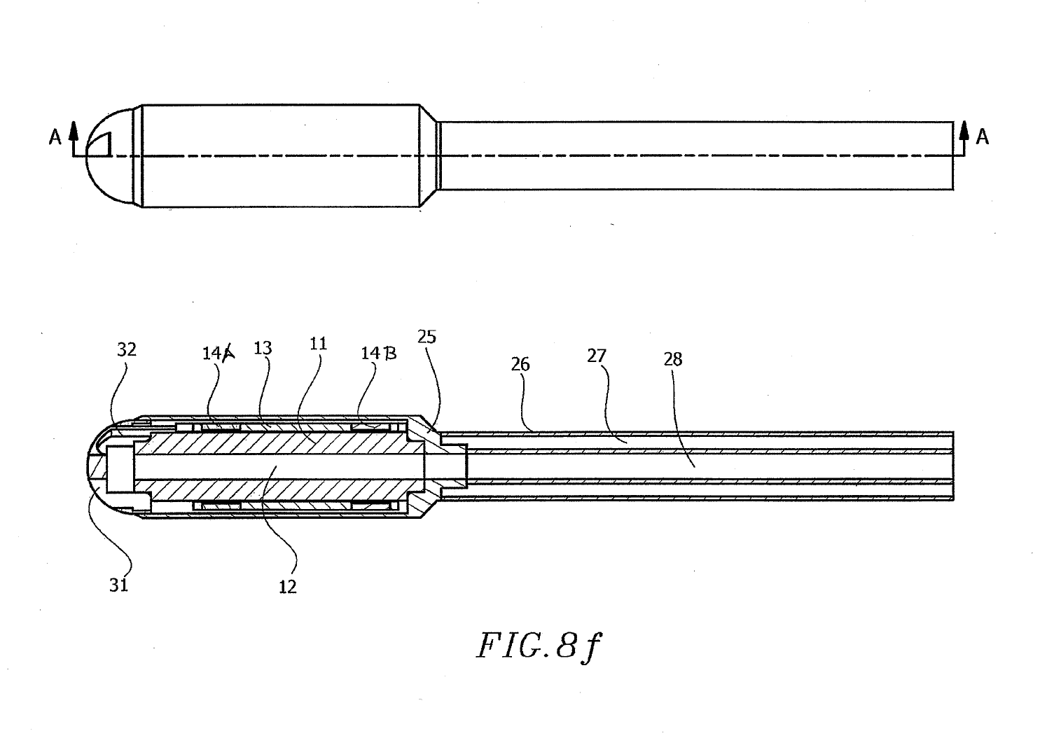

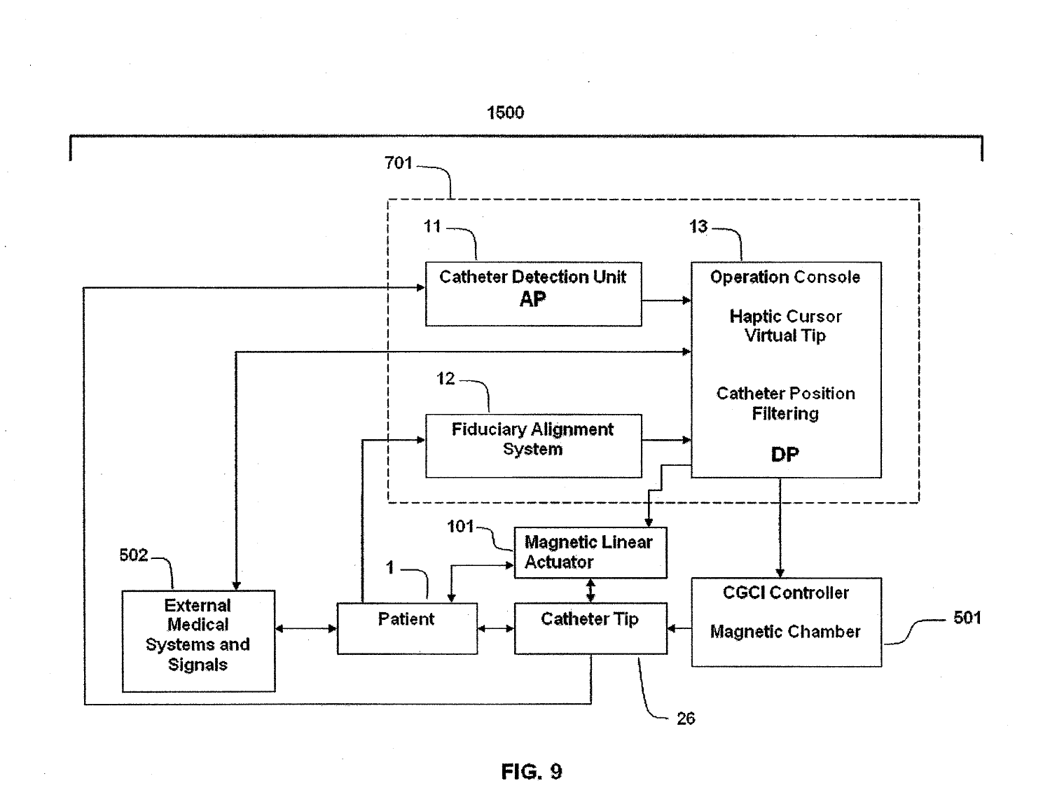

FIG. 8A shows another embodiment where the tool deployed on the distal tip of the catheter 26 is a biopsy tool. The biopsy tool comprises a round distal medical tool housing 31 with a needle element 32 for taking samples of tissue and other biological material. The needle element is directly coupled to the linear actuator 101 (seen in FIG. 8E) without the use of an actuator arm. The round distal medical tool housing 31 provides a smooth surface for the catheter to rest and push up against the desired sample area which allows the needle element 32 to extend out from the medical tool housing 31 and puncture into the tissue or other biological material.

FIG. 8B shows the biopsy tool when in use and while it is being activated by the linear actuator 101. When there is current running through the linear actuator 101, the needle element 32 is extended beyond the surface of the distal medical tool housing 31. When the electric current is reversed, the linear actuator 101 retracts the needles 32 into a “closed” position flush with the distal medical tool housing. The needles 32 may extend as far as 2.5 mm (or more) from the end of the distal medical tool housing 31.

FIGS. 8C and 8D show the device in various stages of deconstruction. FIG. 8C depicts the device with the actuator assembly sheath 25 pulled back from the linear actuator 101. The actuator assembly sheath 25 is, in one embodiment, made of medical grade PVC. FIG. 8D further shows each component of the biopsy tool and its positional relationship to the various parts of the linear actuator 101 including the medical tool housing 31 which provides a nesting area for the needle element. The medical tool housing 31 is, in one embodiment, made out of Teflon or other inert material (e.g., plastics, metals, etc.).

FIGS. 8E and 8F are longitudinal cross sections of the needle element 32 and linear actuator 101 in the “open” or actuated position, and in the “closed” or un-actuated position respectively. FIGS. 8E and 8F additionally depict that the catheter 26 has multiple lumens, namely wire tunnels 27 for housing the wires that apply electric current to the coils 14, and a fluid and vacuum tunnel 28 for transferring fluid to and from the device.

FIG. 9 is a block diagram of a preferred embodiment that incorporates the magnetically-controlled linear actuator end-effecter tool 21 onto a magnetically-guided catheter 26 within a Catheter Guidance Control and Imaging system (CGCI) 1500.

The CGCI unit 1500 includes a magnetic chamber 501, an adaptive regulator, a joystick haptic device for operator control, and a method for detecting a magnetically-tipped catheter 26 is described in U.S. Pat. No. 7,280,865 titled “System and Method for Radar Assisted Catheter Guidance and Control”, U.S. patent application Ser. No. 11/140,475 titled “Apparatus and Method for Shaped Magnetic Field Control for Catheter, Guidance, Control and Imaging.”, U.S. patent application Ser. No. 11/331,994 titled “Apparatus and Method for Generating a Magnetic Field”, U.S. patent application Ser. No. 11/331,485 titled “System and Method for Magnetic Catheter tip,” U.S. patent application Ser. No. 10/621,196 titled “Apparatus for Catheter, Guidance, Control, and Imaging”, U.S. patent application Ser. No. 11/331,781 titled “System and Method for Controlling Movement of a Surgical Tool”, and U.S. patent application Ser. No. 11/697,690 titled “Method and Apparatus for Controlling Catheter Positioning and Orientation” all of which are hereby incorporated by reference. The above magnetic navigation system 1500 is further augmented by the magnetic linear actuator 101 so as to improve the efficiency and utility of the CGCI magnetic chamber 1500 which enables the embodiments of the magnetic linear actuator 101 and catheter tip 26 to perform the intended functions as noted above in the current application.

The CGCI imaging and synchronizations system 701 determines the actual position (AP) of the tool within the patient 1, and specifies the desired position (DP) wherein to guide the magnetically-tipped catheter 26. The CGCI controller 501 employs its magnetic chamber to guide the magnetically-tipped catheter 26 from AP to DP in a closed-loop regulated mode, as to deliver the tool to the desired location within the patient. The CGCI catheter detection unit 11 determines that the tool is at the proper location by using the CGCI fiduciary alignment system 12 to normalize the CGCI detection unit data with the patient's position and orientation. The external medical systems 502 provide the corroborating electrophysiological data that assures the physician that the tool is situated at the desired location. The CGCI operation console 13 is then used to issue commands to the magnetic linear actuator 101 by the standard communications interface.

Also envisioned but not shown are several other embodiments for various medical tools to be deployed on the distal tip of a catheter and actuated by the magnetically controlled linear actuator. These various embodiments include a rotating cleaner tool and a mapping and ablation tool.

In the rotating cleaner tool embodiment, two titanium blades and two “C” shaped permanent magnets are coupled to the bobbin 13. As the external magnetic field rotates around the surgical volume, the “C” magnets will follow accordingly, thus causing the bobbin 13 and blades to rotate and clean the inside of the surgical volume. The blades may be rotated by a variable force with a maximum value of 35 grams.

The final embodiment involving the mapping and ablation catheter involves a MOSFET sensor and RF ablation antennas coupled to the bobbin 13 along with two titanium blades and two “C” shaped magnets. When the external magnetic field rotates around the surgical volume, the “C” magnets will follow accordingly thus causing the bobbin 13, blades, antennas, and sensor to rotate and effectively map and ablate the interior of the surgical volume. Typically, the device employs eight sensors and antenna arms to perform cardiac mapping.

Many alterations and modifications may be made by those having ordinary skill in the art without departing from the spirit and scope of the inventions. Therefore, it must be understood that the illustrated embodiment have been set forth only for the purposes of example and that it should not be taken as limiting the invention as defined by the following invention and its various embodiments.

Therefore, it must be understood that the illustrated embodiment have been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is, therefore, contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

What is claimed is:

1. An apparatus for moving a medical tool on the distal tip of a catheter while in the body of a patient comprising:
a permanent magnet;
a bobbin enclosing the permanent magnet and which is free to slide along the surface of the magnet;
at least two separate coils of electrical wire wound around the bobbin; and
an actuator arm coupled to the bobbin that translates movement of the bobbin into movement of the medical tool.

2. The apparatus of claim 1 wherein the permanent magnet further comprises a hollow core.

3. The apparatus of claim 2 wherein the two separate coils of electrical wire are separately coupled to an external power source to provide a different amount or direction of electric current in each coil when the bobbin moves.

4. The apparatus of claim 3, further comprising a controller for controlling and adjusting the direction and amount of current flow being driven through each independently powered coil that is wound around the bobbin.

5. The apparatus of claim 3 wherein the two separate coils of electrical wire each comprise at least 125 turns of wire.

6. The apparatus of claim 3 wherein the medical tool comprises a shears tool comprising:
a cutting blade coupled to the actuator arm; and
a fixed lower gripping element.

7. The apparatus of claim 3 wherein the medical tool comprises a forceps tool comprising:
an upper gripping element coupled to the actuator arm; and
a fixed lower gripping element.

8. The apparatus of claim 3 wherein the medical tool comprises a biopsy tool comprising:
a fixed round distal housing unit; and
at least two needle-like elements coupled directly to the bobbin.

9. The apparatus of claim 3, further comprising a sheathing that encloses the bobbin and coils.

10. The apparatus of claim 3 wherein the catheter further comprises multiple lumens specifically for providing separate pathways for each wire that is coupled to each coil wound around the bobbin and for the transference of fluid.

11. The apparatus of claim 3, further comprising a catheter guidance control and imaging system.

12. A method for magnetically moving a medical tool deployed on the distal tip of a catheter while in the body of a patient comprising:
detecting the position and orientation of the medical tool in the patient using a position detection system;
applying two variable electric currents to two separate coils of wire;
creating a change in the magnetic flux enclosed by a bobbin on which the coils are wound;
transducing the change in flux into a mechanical force coupled to the medical tool; and
sliding the bobbin and coils distally and proximally in response to the force to move the medical tool that is coupled to the bobbin via an actuator arm.

13. The method of claim 12, further comprising controlling the degree of movement of the medical tool by repetitively adjusting and manipulating the direction and amount of current flow being driven through each independently powered coil that is wound around the bobbin.

14. The method of claim 12 wherein detecting the position and orientation of the medical tool using a position detection system further comprises incorporating the apparatus into a catheter guidance control and imaging system.

15. The method of claim 12 further comprises translating the linear movement of the bobbin into a rotational torque which moves a shears tool that is coupled to the bobbin via an actuator arm and a combination of hinges and pins.

16. The method of claim 12 further comprises translating the linear movement of the bobbin into a rotational torque which moves a forceps tool that is coupled to the bobbin via an actuator arm and a combination of hinges and pins.

17. The method of claim 12 further comprises translating the movement of the bobbin into movement of at least two needle-like elements that are coupled directly to the bobbin.
